(19) United States
(12) Patent Application Publication
(10) Pub. No.: US 2024/0290435 A1
(43) Pub. Date: Aug. 29, 2024

(54) Knowledge Lens for Multidimensional Domains

(71) Applicant: Bristol-Myers Squibb Company, Princeton, NJ (US)

(72) Inventor: Sameen Mayur Desai, Morris Plains, NJ (US)

(73) Assignee: Bristol-Myers Squibb Company, Princeton, NJ (US)

(21) Appl. No.: 18/584,618

(22) Filed: Feb. 22, 2024

Related U.S. Application Data
(60) Provisional application No. 63/487,441, filed on Feb. 28, 2023.

(51) Int. Cl. G16B 50/10; G06F 40/40; G16B 45/00; G16B 50/30

(52) U.S. Cl. CPC G16B 50/10; G06F 40/40; G16B 45/00; G16B 50/30

(57) ABSTRACT

A method includes receiving multidimensional health data from at least one data source. The multidimensional health data includes unstructured data. The method also includes annotating the unstructured data to generate annotated data, processing the annotated data to obtain training healthcare data, and training a knowledge graph on the training healthcare data. The method also includes receiving a query requesting information associated with the knowledge graph and obtaining, from the knowledge graph, the information requested by the query.

### CROSS REFERENCE TO RELATED APPLICATIONS

This U.S. patent application claims priority under 35 U.S.C. § 119(e) to U.S. Provisional Application No. 63/487,441, filed on Feb. 28, 2023. The disclosure of this prior application is considered part of the disclosure of this application and is hereby incorporated by reference in its entirety.

### TECHNICAL FIELD

This disclosure relates to a knowledge lens for multidimensional domains.

### BACKGROUND

Public and private pharmacological data can be obtained from a number of sources. A number of sources may also store statistical information on adverse events to drugs, drug combinations, and concomitant drugs. As the pharmacological data and adverse event data represent multidimensional health data stored across various sources and in formats not amendable to searching, there lacks an ability to link the multidimensional data in a manner suitable to make contextual inquiries on the multidimensional data.

Pharmacodynamics describes how particular treatment drugs affect a disease while pharmacokinetics describes how a body processes a drug. While a pathway for drug intervention is usually well known, pharmacokinetics must be able to consider the pathway that metabolizes the drug itself, and other pathways that drugs may inadvertently and adversely affect. As such, drug safety is an important aspect to consider in the development of new drugs and/or the development of drug combination therapies for the treatment of particular diseases. To further compound the ability to make drug safety predictions, patients exhibiting certain characteristics may be prone to adverse events while treated with certain drugs, drug classes, and/or drug combination therapies, while patients not exhibiting these characteristics are not prone to the adverse events. Accordingly, different sub-classes of a population metabolize drugs differently to provide a variety of potential reactions to a drug which can impact the dosage, safety, and efficacy of that drug and its usefulness for individual patient treatment.

### SUMMARY

One aspect of the disclosure provides a computer-implemented method executed on data processing hardware that causes the data processing hardware to perform operations that include receiving multidimensional health data from at least one data source. The multidimensional health data includes unstructured data. The operations also include annotating the unstructured data to generate annotated data, processing the annotated data to obtain training healthcare data, and training a knowledge graph on the training healthcare data. The operations also include receiving a query requesting information associated with the knowledge graph and obtaining, from the knowledge graph, the information requested by the query.

Implementations of the disclosure may include one or more of the following optional features. In some implementations, the query includes a natural language query and obtaining the information requested by the query includes: processing, using an inference model, the natural language query by performing query interpretation on the natural language query to determine a type of the information requested by the natural language query; and retrieving the information from the knowledge graph based on the type of the information requested by the natural language query. In these implementations, the operations may also include generating, using the inference model, a natural language summary of the information retrieved from the knowledge graph and providing the natural language summary of the information for output from a user device. Here, the inference model may leverage a large language model to generate the natural language summary of the information. Additionally or alternatively, the inference model may include a neural network model.

In some examples, the operations also include receiving canonical reference date. In these examples, annotating the unstructured data includes annotating the unstructured data based on the canonical reference data. In some additional examples, the operations also include receiving concepts that define an ontology for semantically linking the training healthcare data. In these additional examples, training the knowledge graph on the training healthcare data includes using the concepts to train the knowledge graph on the training healthcare data. The information requested by the query may optionally include information regarding a safety of a specific drug for treating a disease.

In some implementations, the operations also include executing a knowledge controller that is configured to display, on a screen of a user device, a user interface for viewing the information obtained from the knowledge graph. In these implementations, receiving the query may include receiving the query from the user device. Here, the user inputs the query through the user interface. Additionally or alternatively, the operations may display the knowledge graph in the user interface as an interactive knowledge graph.

Another aspect of the disclosure provides a system that includes data processing hardware and memory hardware storing instructions that when executed on the data processing hardware causes the data processing hardware to perform operations. The operations include receiving multidimensional health data from at least one data source. The multidimensional health data includes unstructured data. The operations also include annotating the unstructured data to generate annotated data, processing the annotated data to obtain training healthcare data, and training a knowledge graph on the training healthcare data. The operations also include receiving a query requesting information associated with the knowledge graph and obtaining, from the knowledge graph, the information requested by the query.

Implementations of the disclosure may include one or more of the following optional features. In some implementations, the query includes a natural language query and obtaining the information requested by the query includes: processing, using an inference model, the natural language query by performing query interpretation on the natural language query to determine a type of the information requested by the natural language query; and retrieving the information from the knowledge graph based on the type of the information requested by the natural language query. In these implementations, the operations may also include generating, using the inference model, a natural language summary of the information retrieved from the knowledge graph and providing the natural language summary of the information for output from a user device. Here, the inference model may leverage a large language model to generate the natural language summary of the information. Additionally or alternatively, the inference model may include a neural network model.

In some examples, the operations also include receiving canonical reference date. In these examples, annotating the unstructured data includes annotating the unstructured data based on the canonical reference data. In some additional examples, the operations also include receiving concepts that define an ontology for semantically linking the training healthcare data. In these additional examples, training the knowledge graph on the training healthcare data includes using the concepts to train the knowledge graph on the training healthcare data. The information requested by the query may optionally include information regarding a safety of a specific drug for treating a disease.

In some implementations, the operations also include executing a knowledge controller that is configured to display, on a screen of a user device, a user interface for viewing the information obtained from the knowledge graph. In these implementations, receiving the query may include receiving the query from the user device. Here, the user inputs the query through the user interface. Additionally or alternatively, the operations may display the knowledge graph in the user interface as an interactive knowledge graph.

The details of one or more implementations of the disclosure are set forth in the accompanying drawings and the description below. Other aspects, features, and advantages will be apparent from the description and drawings, and from the claims

### DESCRIPTION OF DRAWINGS

FIG. 1 is a schematic view of a system including a knowledge graph linking multidimensional health data and a user interface for viewing the knowledge graph and/or viewing inferences from the knowledge graph.

FIG. 2A is a schematic view of an example knowledge graph builder for constructing the knowledge graph from the multidimensional health data of FIG. 1.

FIG. 2B is a schematic view of an example knowledge controller that receive the multidimensional health data stored across the various data sources via the data input and runs a knowledge graph builder for creating the knowledge graph of FIG. 1.

FIG. 3A is a schematic view of annotated data pertaining to a case narrative for a patient/participant in a clinical trial.

FIG. 3B is a schematic view of annotated data pertaining to a drug label for a particular drug.

FIG. 4 is a schematic view of an inference model receiving a query for information and obtaining the information from a knowledge graph.

FIG. 5A is a schematic view of an example user interface for presenting data represented by a knowledge graph.

FIG. 5B is a schematic view of an example interactive knowledge graph.

FIG. 6 is a flowchart of an example arrangement of operations for a method of creating a knowledge graph from multidimensional health data and running an inference on the knowledge graph.

FIG. 7 is a schematic view of an example computing device that may be used to implement the systems and methods described herein.

Like reference symbols in the various drawings indicate like elements.

### DETAILED DESCRIPTION

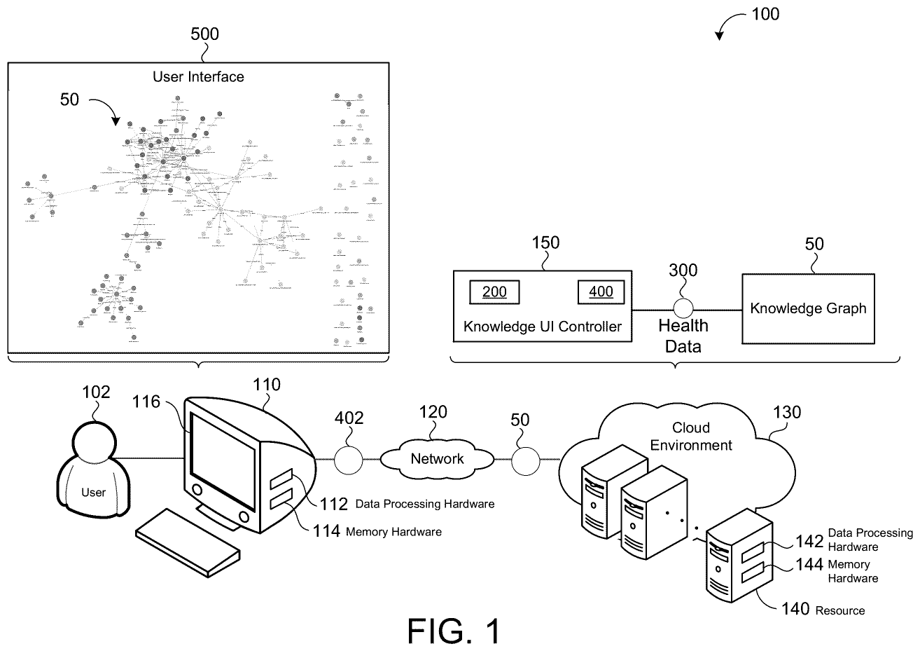

Referring to FIG. 1, in some implementations, an example system 100 includes a knowledge graph 50 linking multidimensional health data 300 and a user interface (UI) 500 for viewing the knowledge graph 50 and/or viewing inferences from the multidimensional health data 300 of the knowledge graph 50. For instance, the knowledge graph 50 may provide insight into potential signals represented by the knowledge graph 50 and have the capability to generate a hypotheses/evidence regarding the occurrence of certain side effects based on pharmacological or biological data. In some examples, an inference model 400 runs on top of the knowledge graph 50 to ensure safety of a drug or drug class for treatment of a disease. As will become apparent, the inference model 400 may receive queries regarding the safety of the drug or drug class with respect to a particular sub-population of patients and make inferences/predictions for the safety of the drug or drug class with respect to the particular sub-population of patients by traversing the knowledge graph 50. For instance, the inference model 400 may predict adverse events for a particular drug, and even more specifically, predict adverse events for a particular drug with respect to a population of patients having a specific character trait. The predictions may include probabilities or likelihoods.

The inference model 400 may additionally enable search functionality through the knowledge graph 50 to gain insights on the information represented by the knowledge graph 50. That is, the inference model 400 may extend the knowledge graph 50 to do highly relevant search across multimodal data contained in the knowledge graph 50 in order to bring statistics, data, inferences, and/or recommendations related to a safety profile of a drug, drug class, and/or population or sub-population of patients. In this manner, search results may highlight similarities between drug candidates and established drugs from a safety perspective through the use of various similarity algorithms including, but not limited to, embeddings, sine/cosine/jaccard similarities, or other types of distance measures between data points in the knowledge graph 50.

As will become apparent, the generation of the knowledge graph 50 and the ability to interact with the knowledge graph 50 provides a multitude of operational use cases within pharmacovigilance such as providing an ability to understand duplicates, case management operations, and medical review of not only individual cases, but aggregates of cases as well. The insights provide a reduction in complexity, cost, and time of integration and migration of safety information related to a drug, drug class, and/or population or sub-population of patients. The UI 500 provides an extensible framework for analysis of PV data to enable prospective views using retrospective data represented by the knowledge graph 50. In this manner, a concept of a patient journey through treatment of a drug or drug class can be generated/predicted and provide a fundamental change in how adverse events can be learned within the system, and thereby shift the heart of the underling process of understanding safety information from the specific case itself to the patient.

The system 100 includes a user device 110 associated with a user 102 and in communication with a remote system 130 via a network 120. The user 102 may include, without limitation, a research professional, a clinical trial professional, a physician, a healthcare provider, or a patient. The user device 110 corresponds to a computing device, such as, without limitation, a desktop workstation, a laptop workstation, or a mobile computing device (e.g., smart phone or tablet). The remote system 130 may be a distributed system (e.g. a cloud environment) having scalable/elastic resources 140 including computing resources 142 (e.g., data processing hardware) and storage resources 144 (e.g., memory hardware). The computing resources 142 may include a service abstraction layer and a hypertext transfer protocol wrapper over a server virtual machine instantiated thereon. As such, the computing resources 142 may be configured to receive queries 402 from the user device 110 and send responses (e.g., the knowledge graph 50, portions of the knowledge graph 50, predictions inferred from the knowledge graph 50 by the inference model, etc.) to the user device 110.

In the example shown, the computing resources 142 manage storage of the knowledge graph 50 on the storage resources 144. The computing resources 142 may further execute a knowledge controller 150 that is configured to communicate with the user device 110 and act as an interfacing mechanism for enabling the user device 110 to build/create the knowledge graph 50, interact with the knowledge graph 50, and perform operations (e.g., read/write) on the knowledge graph 50. Specifically, the knowledge controller 150 may run a knowledge graph builder 200 to enable input of the multidimensional health data 300 and rules/ontologies for identifying particular concepts/entities in the health data. The knowledge graph builder 200 may then build/create the knowledge graph 50 such that the knowledge graph 50 represents each concept identified in the health data as a node and links related nodes together based on interrelationships between the concepts. As such, the knowledge graph 50 may represent clusters of cases, wherein each case includes a group of related nodes linked to one another based on the interrelationships between the concepts represented by the nodes. For instance, a case may include a patient node representing a patient having a medical condition, one or more drug nodes each representing a drug or drug class prescribed to the patient for treating the medical condition, and one or more adverse event (AE) nodes each representing an AE experienced by the patient while prescribed the drugs or drug classes. Described in greater detail below, some cases in the knowledge graph 50 may additionally or alternatively include nodes representing other types of concepts that may be of interest as specified by the rules/ontologies input to the knowledge graph builder 200.

Once the knowledge graph 50 is built by the knowledge graph builder 200, the knowledge controller 150 enables data retrieval of the knowledge graph 50 from the storage resources 144 and displays the UI 500 on a screen 116 of the user device 110 for viewing the knowledge graph 50. The knowledge controller 150 may permit the user 102 to interact with the knowledge graph 50 displayed in the UI 500. For instance, the user 102 may select nodes of interest to ascertain more detailed information about the selected node. In one example, the user 102 may select a patient node and the knowledge controller 150 may cause the UI 500 to present a pop-up window that presents detailed information pertaining to the patient represented by the patient node. The detailed information may include the patient's demographics (i.e., age, gender, residence, etc.), biomarkers, diseases, prescribed medications, treating physicians, or any other characteristic of the patient. The knowledge controller 150 may additionally allow the user 102 to provide queries 402 to present specific data from the knowledge graph 50 that is of interest. For instance, the user 102 may provide a single natural language query or multiple individual queries that request the knowledge controller 150 to present cases from the knowledge graph 50 that include 50 to 60 year-old males who were prescribed a particular drug combination. In this example, the knowledge controller 150 may update the knowledge graph 50 so that only the linked nodes of the cases that include 50 to 60 year-old males prescribed the particular drug combination are presented for display in the UI 500 while all cases are excluded from being displayed in the UI 500.

In some implementations, the knowledge controller 150 executes the inference model 400 to make inferences/predictions from the knowledge graph 50 with respect to information requested by queries 402 input by the user 102. For instance, the user 102 may input a natural language query requesting information regarding the safety of a specific treatment drug with respect to a specific patient character trait (e.g., 50-60 year-old male) and the inference model 400 may make inferences/predictions for the safety of the treatment drug with respect to the character trait by traversing the knowledge graph 50. In some examples, the inference model 400 generates a natural language summary based on the inferences/predictions for the safety of the treatment drug with respect to the character trait specified by the query. In this example, the summary may indicate “There is a high likelihood that a male between the ages of 50 and 60 will experience circulatory collapse if prescribed the treatment drug”.

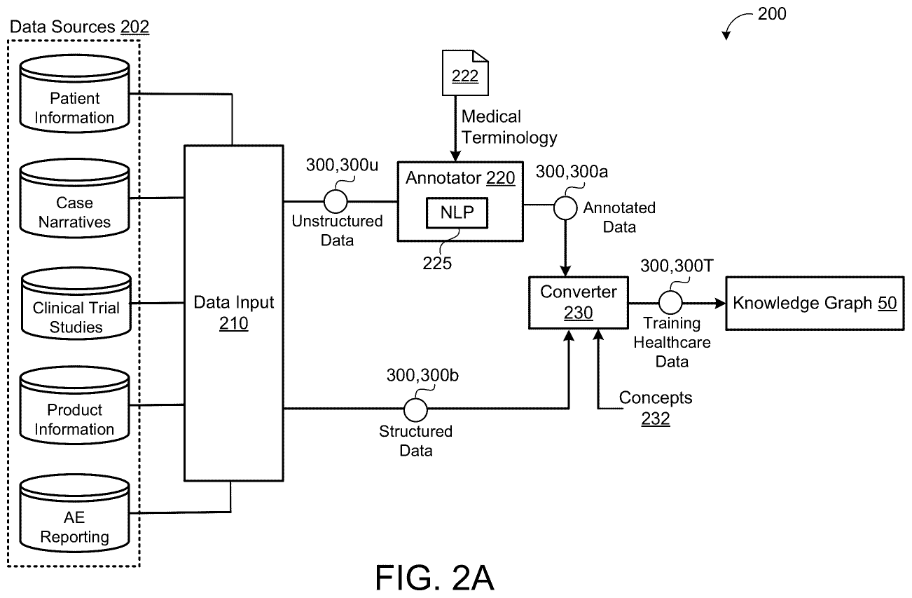

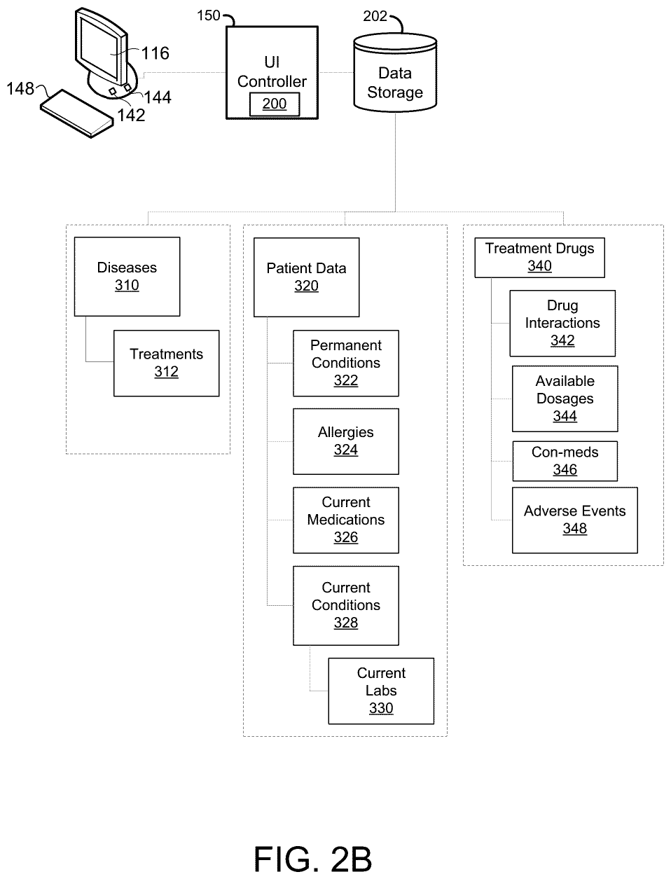

FIG. 2A shows a schematic view of an example knowledge graph builder 200 for use in creating the knowledge graph 50 from the multidimensional health data 300 stored across various data sources 202. The knowledge graph builder 200 includes a data input 210 that receives the data 300 from the data sources 202. In some examples, the user 102 uses the knowledge controller 150 to retrieve the multidimensional health data 300 from the data sources 202 by allowing the user 102 to provide criteria for the type of multidimensional health data 300 to be represented by the knowledge graph 50. The knowledge graph builder 200 uses the multidimensional health data 300 as training data for training the knowledge graph 50.

In the example shown, a non-exhaustive list of data sources 202 is depicted. The data sources 202 may be interchangeably referred to as ‘data stores 202’. Details of the present disclosure may include other data sources 202 for providing the multidimensional health data 300 in addition to, or in lieu of, any of the data sources 202 depicted in the example shown. The data sources 202 include a patient information data source, a case narratives data source, a clinical trial studies data source, a product information data source, and an adverse event (AE) data source. The patient information data source may include health data for each of a corpus of patients. Patients in the corpus may be participants across various clinical trials and/or studies related to the treatment of diseases, as well as to aid in the development of drug treatment therapies for treatment of those diseases. The patient information for each patient in the corpus of patients may include structured and/or unstructured data including patient notes entered into one or more electronic medical records (EMRs) by a research professional, a clinical trial professional, a physician, and/or a healthcare provider. The patient data/notes may include demographic information for each patient such as, without limitation, the patient's age, gender, ethnicity, height, weight, and body mass index (BMI), as well as genetic data, phenotypic, proteome, climate, drug adverse event history, any diseases/conditions, allergies, prior health conditions, vital signs, recommended treatments, risks, medical history, family health history, lab results, current medications, and/or past medications. Source data for drug adverse event history and/or medical history may be acquired by accessing, soliciting, or assembling data on patients experiencing adverse drug reactions, and comparing the data against data from a control set of a broad population who are not taking the drug/drugs in question in order to see the relationship between certain reactions and genotype/phenotype. For example, light skinned people (a kind of phenotype with genotypic background) are generally prone to sunburn and may additionally be particularly sensitive to certain drugs. Population genetics information includes a wide variety of sources including DNA samples solicited directly from people who have had documented adverse reactions to certain drugs.

The patient information for one or more patients in the corpus of patients may also include identifiers and details for any clinical trials and/or studies (past or present) that the patients participated in, as well details of outcomes from the trials and adverse events experienced by the patients. Patient notes input in the form of unstructured data may include numerous strings of characters arranged into sentences. The sentences may be organized in one or more paragraphs.

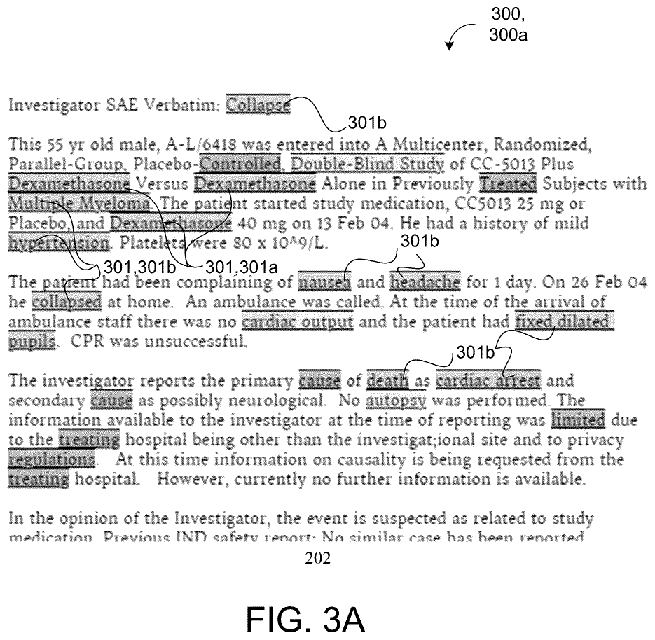

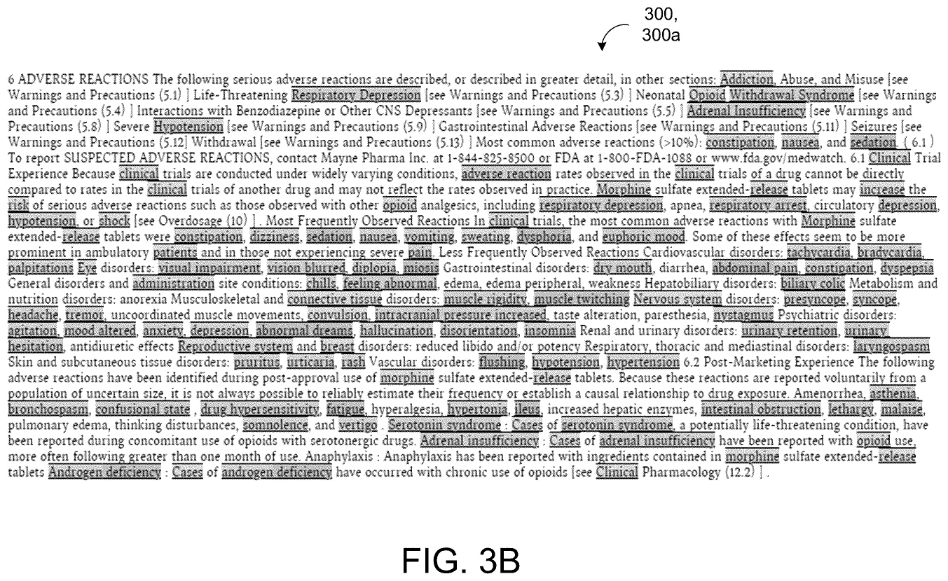

The case narrative data store may include narratives for patients participating in a clinical trial or other health study. Case narratives may be stored in the form of unstructured data including numerous strings of characters arranged into sentences. An example case narrative is depicted in FIG. 3A. As used herein, the case narrative depicting narrative of a clinical trial is exemplary only, and the present disclosure may similarly include published literature and scientific research articles as other types of unstructured data including numerous strings of characters arranged into sentences. The sentences may be organized in one or more paragraphs. Each case narrative may follow regulatory requirements and follow procedures aimed at reducing the burden of time and cost for effectively reporting patient safety during all phases of clinical studies, whether conducted in healthy volunteers or in patients with the disease/condition under study. A patient safety narrative provides a full and clinically relevant, chronological account of a progression of an event experienced during or immediately following a clinical study. Case narratives may follow Council for International Organizations of Medical Sciences (CIOMS) forms; Case Report Forms (CRFs); MedWatch forms, Data Clarification Forms (DCFs), and clinical database listings. In some examples, the case narrative data store includes Clinical Study Reports (CSRs) that contain brief narratives describing each death, each other serious AE, and other significant AEs that are judged to be special interest because of clinical importance. As such, the narratives contained in a CRS should include the nature, intensity, and outcome of an AE; the clinical course leading to the AE; an indication of timing relevant to study drug administration; relevant laboratory measures; action taken with the study drug (and timing) in relation to the AE; treatment or intervention; post-mortem findings (if applicable); investigator's and sponsor's (if appropriate) opinion on causality; patient identifier, age and sex of patient; general clinical condition of patient, if appropriate; disease being treated with duration of current episode of illness; relevant concomitant/previous illnesses with details of occurrence/duration; relevant concomitant/previous medication (e.g., concomitant drugs or con-meds) with details of dosage; and test drug administered, including dose and length of time administered. A patient safety narrative in, or appended to, a CSR describes all relevant events for a single patient, with relevant background information as detailed above. An individual CSR (ICSR) concerns one patient, one or more identifiable reporters, one or more suspected AEs that are clinically and temporally associated with treatment, and one or more suspected medicinal products. In the context of a clinical trial, an individual case is the information provided by a primary source to describe a serious adverse event related or unrelated to the administration of one or more investigational medicinal products to an individual patient at a particular point of time. The AE reported should be the diagnosis. If a diagnosis has not been made at that time, the case may contain several signs and symptoms instead, and therefore, more than one reported event. ICSRs prepared post-marketing can differ from this in that several event terms may be reported in a single case; these events should be temporally or clinically associated, and they will be ordered according to clinical relevance for the product, i.e., a serious unexpected AE would be designated the “primary event” for reporting purposes, whereas non-serious or expected AEs would be ranked lower within the case. Furthermore, in post-marketing ICSRs, all spontaneous reported AEs are considered related to the medicinal product unless specified otherwise by the reporter, whereas in a clinical setting, the investigator makes his/her interpretation as to causality.

The clinical trial studies data source may include one or more regulatory sources of accessible information on publicly and/or privately supported clinical studies on a wide range of diseases and conditions. As such, the clinical trial studies data source may include one or more web-based resources (e.g., www.clinicaltrials.org) that provide patients, their family members, health care professionals, researchers, and the public with easy access to information on publicly and privately supported clinical studies on a wide range of diseases and conditions. Information in these web-based resources may be provided and updated by the sponsor or principal investigator of the clinical study. Studies are generally submitted to the website (that is, registered) when they begin, and the information on the site is updated throughout the study. In some cases, results of the study are submitted after the study ends. In one example, the clinical trials data source includes www.clinicaltrials.gov.

The clinical trial studies data store may contain both clinical and post marketing data about drugs and drug classes used in clinical trials, thereby providing useful safety information for an entire life cycle of a product commencing from its first use with a patient/human. Some of the information stored in the clinical trial studies data store may include the same or substantially the same information as the case narrative data source. Here, the clinical trial studies data store contains information about medical studies in human volunteers. Most of the records stored in the data source describe clinical trials (also called interventional studies). A clinical trial is a research study in which human volunteers are assigned to interventions (for example, a medical product, behavior, or procedure) based on a protocol (or plan) and are then evaluated for effects on biomedical or health outcomes. The clinical trial studies data store also contains records describing observational studies and programs providing access to investigational drugs outside of clinical trials (expanded access). Records for clinical trials may summarize the following types of information: disease/condition being studied/treated; intervention (medical product, behavior, or procedure); title, description, and design of the study; treatment drug or drug combination that is part of the study, concomitant drugs, eligibility requirements for participants in the study; locations where the study is conducted; contact information for the study locations; links to relevant information; description of study participants (the number of participants starting and completing the study and their demographic data); outcomes of the study; and a summary of AEs. Concomitant drugs (also referred to as ‘con-meds’) are other prescription medications, over-the-counter (OTC) drugs, or dietary supplements that a study participant takes in addition to the drug or drug combination under investigation. Con-meds may be used by study subjects for the same indication as the study or for other indications

The product information data source includes a corpus of available drugs and/or products/devices for treating various diseases and conditions. The product information data source may include real world data about the type or class of drug, metabolic pathways, drug pharmacokinetics, and pharmacodynamics. The product information data source may provide drug taxonomies that offer characteristics of drugs including metabolites, clearance rates, peak serum levels, pharmacodynamics, therapeutic category, chemical structure, or a way to group drugs and explore the relationship to both reactions and genotypes. In some examples, the corpus of available drugs includes drugs and drug combinations for treating a particular type of disease, such as available immunotherapy drugs for treating cancer. For each drug in the corpus of available drugs, the product information data source may provide a corresponding drug label used to ensure patient safety by giving healthcare professionals a summary of the safety and efficacy of the corresponding drug. In some scenarios, the drug labels are directed toward a patient population when the drug is an over-the-counter drug. However, in scenarios when a drug is a prescription or investigational drug, the drug label is not aimed at the patient population because prescription and investigational drug administration is always under the supervision of a healthcare practitioner that is licensed to prescribe or otherwise authorize administration of the drug. In general, the following list includes an outline of requirements in a drug label: highlights providing a concise summary of label information; full prescribing information; limitations statement; product names; date of approval in each of one or more jurisdictions; boxed warning; recent major changes; indications and usage; dosage and administration; dosage forms and strengths; contraindications; warnings and precautions; adverse reactions; drug interactions, use in specific populations, and patient counseling information statement. FIG. 3B shows the adverse reactions listed in a drug label for an example drug. Investigational drugs may include one or more of a protocol number, a generic name, a name and address of a sponsor, patient identifier, special warnings, investigator's name, a study's acronym or title, name of an institutional review board (IRB) for the drug, dosage/concentration/strength of investigational drug, formulation (e.g., lyophilized powder, solution, suspension, capsule, tablet, etc.), lot/batch number, expiration/retest date, and an approved standardized identifier that is unique and distinctive from other investigational drugs.

In some examples, the product information data source includes publicly available open product labels maintained by the United States Food and Drug Administration (FDA). The product information data source may include one or more drug code directories, such as the National Drug Code (NDC) Directory maintained by the FDA that includes information about finished drug products, unfinished drugs, and compounded drug products. Here, drug manufacturers/establishments are required to provide a regulator (i.e., the FDA) with a current list of all drugs manufactured, prepared, propagated, compounded, or processed for sale at their facilities. Drugs are identified and reported using a unique, three-segment number called the National Drug Code (NDC) which serves as the FDA's identifier for drugs. The FDA may publish the NDC numbers in the NDC Directory which is updated daily. Whereas drug labels may be recorded in a non-structured format, the drugs submitted to the FDA for inclusion in the NDC Directory are in the form of structured product labeling (SPL) electronic listing files by labelers, who may include a manufacturer or entity named on the product label. The NDC Directory includes the product listing data submitted for all finished drugs including prescription and over-the-counter drugs, approved and unapproved drugs, and repackaged and relabeled drugs.

Moreover, with respect to unfinished drugs such as investigational drugs being investigated in clinical trials, drug manufacturers producing the active pharmaceutical ingredients are required to provide the FDA with a current list of all drugs manufactured, prepared, propagated, compounded or processed in commercial distribution in the U.S. at their facilities. As such, the NDC Directory may maintain an unfinished drug database containing product listing data submitted for all unfinished drugs, including active pharmaceutical ingredients, drugs for further processing, and bulk drug substances for compounding. Notably, the resulting knowledge graph 50 may advantageously link a finished drug to related information for when the finished drug or at least its pharmaceutical ingredients were at the unfinished stage so that the user 102 may readily view (e.g., via the interface 500) relevant information pertaining to the drug during all stages of development.

Additionally, the product information data source may include information about finished compounded human drug products produced by outsourcing facilities that may have elected to assign the NDC to their products. Such outsourcing facilities can be eligible for exemptions from drug registration and listing requirements if they meet certain conditions under law, whereby these outsourcing facilities may, but are not required to, assign NDC numbers to their finished compounded human drug products. The NDC Directory may only contain compounded drug products reported with the marketing category “Outsourcing Facility Compounded Human Drug Product (Exempt from Approval Requirements)” and that were assigned an NDC number. The product information may include search results containing information reported to the FDA within the last two years. Notably, an annotator 220 may annotate data obtained from the NDC Directory related to unfinished drugs and compounded human drug products so that the data when presented in the knowledge graph 50 is distinguishable from finished drugs since mere inclusion of a product in the NDC Directory does not imply that the FDA has verified the information provided or that the products are FDA approved. In this situation, the annotator 220 may view a label/tag in the corresponding structured product labeling (SPL) when the product was submitted.

The AE reporting data source includes records of all AE cases reported across one or more regulatory authorities. The AE reporting data source may include adverse events provided, for example, from pharmaceutical corporations, hospitals, physicians, health insurers, and state, federal and international agencies. A primary source of pharmaceutical industry data is the individual adverse events recorded by the various pharmaceutical corporation safety departments. In each case, source data may be focused on clinical trials, post-market surveillance, research databases, or the like. Unedited data in each source database is referred to as “verbatim.” Clinical trial data available in literature includes safety data. Other information is collected and can be accessed from the World Health Organization (WHO), the General Practice Research Database (GPRD), and so forth. For instance, the AE reporting data source may include the Food and Drug Administration Adverse Event Reporting System (FAERS) that maintains data for use by the general public to search for information related to human AEs reported to the FDA by the pharmaceutical industry, healthcare providers, and consumers. That is, the AE reporting data source may contain data on AEs reported to a regulatory authority (e.g., the FDA) on a particular drug or biologic product. However, as the reports do not indicate that the particular drug or biologic caused the AE, the data maintained by the AE reporting data source by itself is not an indicator of a safety profile of the drug or biologic product. However, the data maintained by the AE reporting data source may include limitations of containing duplicate and incomplete reports where some reports may be missing necessary information, contain existence of reports that do not establish causation of the AE and the drug or biologic product since the information in the reports reflects only the observations and opinions of the reporter of the AE, contain information in reports that have not been verified or medically confirmed, and provide no ability to establish rates of occurrence with the reports. As will become apparent, the creation of the knowledge graph 50 based upon the multidimensional health data 300 collected from all of the various data sources 202 and in conjunction with the knowledge controller 150 can provide an ability to understand safety of drugs with respect to particular sub-populations and characteristics of the sub-populations in a manner that would not be possible by simply searching the AE reporting data source.

With reference to FIG. 2B, the knowledge controller 150 may be configured to execute instructions to receive the multidimensional health data 300 stored across the various data sources 202 via the data input 210 (FIG. 2A) and run the knowledge graph builder 200 for creating and updating the knowledge graph 50. The health data 300 received via the data input 210 may be stored on the memory hardware 114 of the user device 110 and/or the memory hardware 144 of the cloud computing environment 130. In some examples, the multidimensional health data 300 is classified into one of three categories: (i) disease data 310; (ii) patient data 320; and (iii) treatment drug data 340. These categories of multidimensional health data 300 are exemplary only and may additionally or alternatively include other categories such as those representing payer data including claims and prescriptions. The disease data 310 may include a list of diseases and conditions each having a list of one or more treatments 312. The treatments can be accepted treatments of drugs or drug combinations as well as past and present experimental treatments conducted via clinical trials. The patient data 320 may be stored as a table containing data permanently associated with each individual patient, such as identification, demographics, and a plurality of sub-tables 322, 324, 326, 328, 330 linked to the table in a few-to-many relationship, whereby data related to each record of information in the table of the patient data 320 is stored in the various sub-tables corresponding to the record. For instance, sub-table 322 may list permanent medical conditions of the patient, sub-table 324 may list known allergies of the patient, sub-table 326 may list all current medications the patient takes, sub-table 328 may list all current conditions the patient is experiencing which may be populated from AE events reported during a clinical trial and/or by an HCP and/or by comparing records (i.e., lab results) of a current labs sub-table 330.

The treatment drug data 340 may be represented by a table including a schedule of all available treatment drugs, drug classes, and drug combinations used for the treatment of diseases. The treatment drug data 340 may be indexed to be linked to a plurality of sub-tables 342, 344, 346, 348. Each drug represented by the treatment drug data 340 may be populated with drug information and scaled guidelines. The drug information may include a respective NDC number, drug class, chemical class, biological pathway, metabolites, structure, any generic names, and a delivery method. The scaled guidelines may indicate known health risks and efficacy for treating an underlying disease/condition. The biological pathway associated with a drug or drug class may indicate which mechanisms, such as enzymes, are activated (i.e., over/under expressed) to lead to a certain biologic activity. That is, a drug may target an enzyme that is instrumental in a particular pathway, yet the pathway can be redundant such that blocking the pathway can strengthen another pathway in a phenomenon known as a signaling cascade which often occurs when targeting pathways for treating cancer. Thus, as cancer implements multiple pathways, drug combination treatments are often required to target multiple enzymes in a given pathway. The sub-table 342 may include a list of drug interactions indicating drugs/medications that are known to interact with the underlying drug. The sub-table 344 may indicate available dosages for the underlying drug and the sub-table 346 may indicate concomitant drugs (e.g., con-meds) that a patient or participant takes in addition to the underlying drug or drug combination.

Notably, the multidimensional health data 300 input to the knowledge graph builder 200 via the data input 210 includes both unstructured data 300u and structured data 300b. The unstructured data 300u may include numerous strings of characters arranged into sentences. The sentences may be organized in one or more paragraphs. Referring back to FIG. 2A, the knowledge graph builder 200 executes the annotator 220 to parse the unstructured data 300u and extract key terms and information therefrom to provide annotated data 300a for use in creating the knowledge graph 50. The annotator 220 may execute one or more natural language processing (NLP) models 225 each configured to receive the unstructured data 300u and output corresponding annotated data 300a. Some NLP models may be trained for annotating particular types of unstructured data 300u. In some examples, a special-purpose NLP model is trained to parse unstructured data 300u pertaining to a case narrative and output annotated data 300a that annotates the case narrative with key terms identified in the case narrative. For instance, FIG. 3A shows annotated data 300a pertaining to a case narrative for a patient/participant in a clinical trial that collapsed while being treated for Multiple Myeloma with an experimental drug in combination with another drug Dexamethasone. In the example shown, the NLP model 225 annotates the case narrative such that different types of terms 301, 301a-d are identified an annotated. Here, a first term 301a is associated with recitations of specific drugs (e.g., Dexamethasone) in the case narrative and a second term 301b is associated with recitations of adverse events (e.g., collapse/collapsed, Multiple Myeloma, hypertension, nausea, headache, fixed dilated pupils, death and arrest) in the case narrative. Other unique types of terms can be identified and annotated in the case narrative by the NLP model 225. The same or a different NLP model 225 may be trained to parse unstructured data 300u pertaining to a drug label and output annotated data 300a that annotates the case narrative with key terms identified in the case narrative. For instance, FIG. 3B shows annotated data 300a pertaining to a drug label for a particular drug whereby the NLP model 225 annotates each instance of an adverse event recited in the corresponding drug label.

Referring back to FIG. 2A, in some implementations, the annotator 220 receives canonical reference data 222 including dictionaries, thesauruses, taxonomies, and hierarchies for use in generating the annotated data 300a from the unstructured data 300u input to the annotator 220. As such, the canonical reference data 222 may not only provide terms that NLP model(s) 225 can use to identify when parsing unstructured data 300u, but may also supplement those identified terms with related terms, synonymous terms, and lexical variants. An example of canonical reference data 222 includes the Medical Dictionary for Regulatory Activities (MedDRA) that identifies a multitude of different adverse events at different hierarchical levels. Here, the MedDRA may include a hierarchy of five levels arranged from very specific to very general, wherein the most specific level, called “Lowest Level Terms” (LLTs) includes more than 80,000 terms which parallel how information is communicated and reflect how an observation might be reported in practice. The next level, called “preferred Terms” (PTs), includes a distinct descriptor (single medical concept) for a symptom, sign, disease diagnosis, therapeutic indication, investigation, surgical or medical procedure, and medical social or family history characteristics. Each LLT is linked to only one PT and each PT has at least one LLT as well as synonyms and lexical variants (e.g., abbreviations, different word order, etc.) of the PT. The next level, called “High Level Terms” (HLTs) groups together related PT's based upon anatomy, pathology, physiology, aetiology or function. HLTs, related to each other by anatomy, pathology, physiology, aetiology or function, are in turn linked to “High Level Group Terms” (HLGTs). Finally, the MedDRA may group HLGTs into the most general level, called “System Organ Classes” (SOCs) which are groupings by aetiology (e.g., infections and infestations, manifestation site (e.g. Gastrointestinal disorders) or purpose (e.g. Surgical and medical procedures). Additionally or alternatively, the canonical reference data 222 may include custom data including rules, terminology, language models, dictionaries, and/or libraries for use by the annotator 220 when parsing and annotating the unstructured data 300u received via the data input 210 into the annotated data 300a. The canonical reference data 222, such as MedDRA, may additionally characterize reported adverse events by their seriousness.

With continued reference to FIG. 2A, the knowledge graph builder 200 also includes a converter 230 that is configured to merge the annotated data 300a and the structured data 300b into training healthcare data 300, 300T for training the knowledge graph 50. The training healthcare data 300T may include data associated with a disease/condition (e.g., cancer), patients and/or participants of clinical trials diagnosed with the disease/condition, treatment classes for treating the disease/condition, various treatment drugs and drug combinations (including both approved and experimental drugs and drug combinations that are the subject of a study/clinical trial) related to the treatment classes that are prescribed to the patients and/or participants, any concomitant drugs that the patients/participants are taking in addition to the underlying treatment drug or drug combination, efficacy of the treatment drugs and drug combinations, and any adverse events experienced by the patients/participants while taking the treatment drugs and drug combinations and/or after the patients/participants stop taking the treatment drugs and drug combinations.

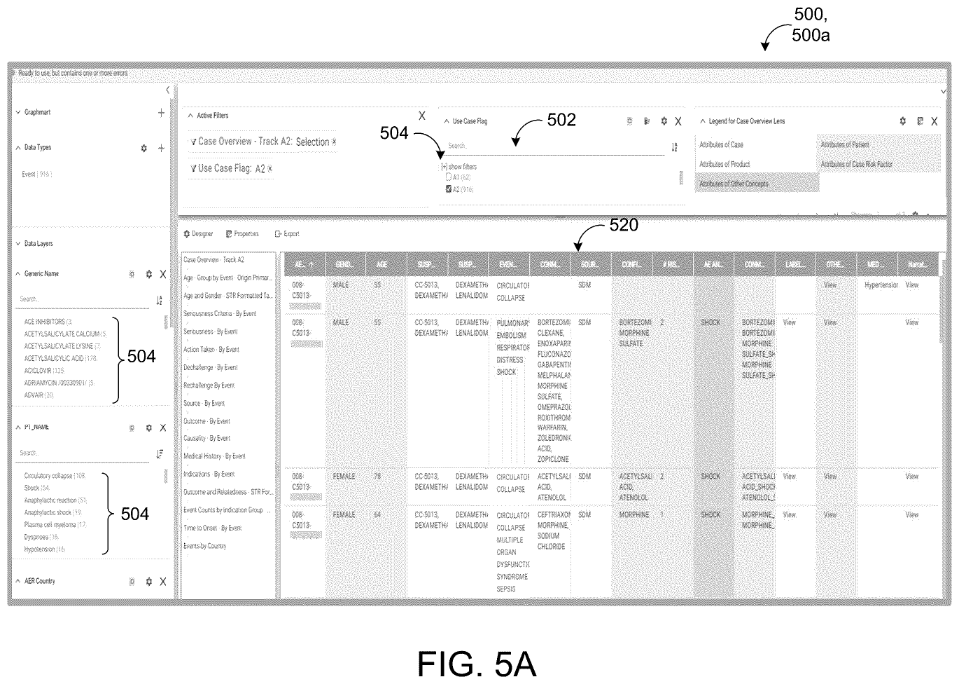

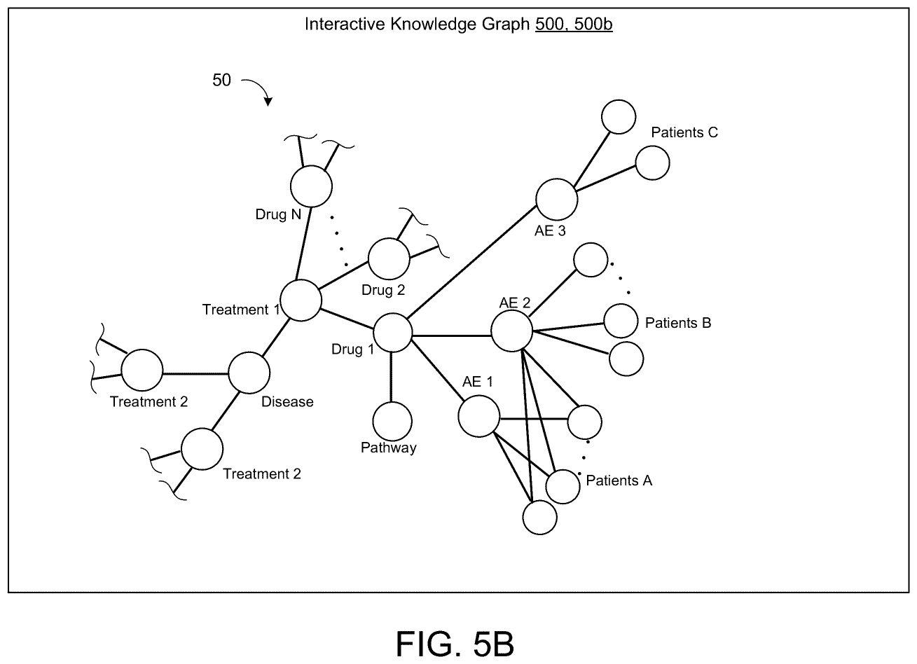

In some examples, the knowledge graph builder 200 uses the training healthcare data 300T to train the knowledge graph 50 to provide a drug safety system capable of making inferences/predictions for the safety of a drug, drug combination, and/or drug class used to treat a disease/condition. The converter 230 may receive concepts 232 that provide an ontology for training the knowledge graph 50 on the multidimensional training healthcare data 300T. Specifically, the concepts 232 allow the converter 230 to semantically link the training healthcare data 300T within the knowledge graph 50 to permit contextual inquiries on the healthcare data 300. The concepts 232 may include user-specified rules that define nodes related to a treatment for a disease and edges or links for connecting the nodes to depict interrelationships (e.g., relations) between the concepts related to the treatment. In some implementations, the knowledge graph 50 generated by the knowledge graph builder 200 is self-forming such that the knowledge graph builder 200 uses the NLP models 225 and canonical reference data 222 to identify and create the concepts/nodes from the healthcare data 300 alone without requiring the user to explicitly provide the concepts 232. Continuing with the example, the concepts 232 input to converter 230 of the knowledge graph builder 200 may define nodes that include a disease node (e.g., cancer or a particular type of cancer such as melanoma), a treatment node (e.g., immunotherapy), drug nodes related to the treatment node (which may indicate biological pathway targeted, adverse event (AE) nodes related to the treatment and drug nodes, a biological pathway node related to the drug nodes, and patient/participant nodes related to the disease, treatment, and drug nodes. The user interface 500, 500b of FIG. 5B depicts a view of an example knowledge graph 50 that the user 102 may interact with.

The resulting knowledge graph 50 represents a model that includes individual concepts (nodes) and predicates that describe properties and/or relationships between those individual nodes. A logical structure (e.g., Nth order logic) may underlie the knowledge graph that uses the predicates to connect various individual nodes. The knowledge graph 50 and the logical structure may combine to form a language that recites facts, concepts, correlations, conclusions, propositions, and the like. The knowledge graph 50 and the logical structure may be generated and updated continuously or on a periodic basis by an artificial intelligence engine (i.e., the knowledge graph builder 200) responsive to new healthcare data 300 received from the data sources 202 at the data input 210 (FIG. 2A). The predicates and individual nodes may be generated based on healthcare data that is input to the knowledge graph builder 200. Updated or new canonical reference data 50 may be continuously provided to the knowledge graph builder 200 to enable the knowledge graph builder 200 to modify the individual elements and predicates represented by the knowledge graph 50 on an ongoing basis.

The converter 230 of the knowledge graph builder 200 may generate the knowledge graph 50 from the training healthcare data 300T and the concepts 232 by determining semantic relationships to align the training healthcare data 300T with the concepts 232. In some examples, the converter 230 utilizes machine learning techniques to align and integrate the training healthcare data 300T into the concepts 232 for generating the knowledge graph 50. Additionally or alternatively, the converter 230 may utilize any combination of schema-level matching techniques, instance-level matching techniques, or hybrid matching techniques to align and integrate the training healthcare data 300 into the concepts 232.

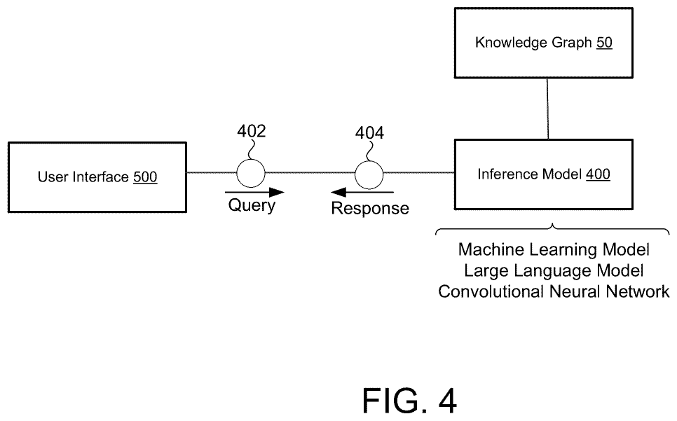

Referring to FIG. 4, the user interface 500 executing on the user device 110 permits the user 102 to issue queries 402 to the inference model 400 that request information associated with the knowledge graph 50. In some examples, the query 402 received from the user device 110 requests the inference model 400 to return safety information associated with a drug, drug class, or other form of treatment (i.e., surgery) used to treat a disease. The user 102 may input the query 402 via the user interface 500 as a natural language query and the inference model 400 is configured to perform query interpretation on the natural language query to determine what type of information the user 102 is requesting from the knowledge graph 50. For instance, the natural language query 402 may include “Return all adverse events reported for investigational drug X” whereby the inference model 400 may convert the natural language query 402 into a graphical query to leverage the existing structure of the knowledge graph 50 and retrieve the requested information. The natural language query 402 may specify different levels of granularity for the information the inference model 400 is requested to return from the knowledge graph 50. For instance, the natural language query 402 may include “Return all adverse events reported for males between the ages of 40 to 55 years diagnosed with melanoma and treated with investigational drug X”. The inference model 400 may return a response 404 conveying the requested information for the user interface 500 to output to the user 102. Here, the user interface 500 may display the response 404 on a display 116 of the user device 110 and/or audibly output synthesized speech through a speaker of the user device 110 that conveys the requested information to the user 102.

In some implementations, the inference model 400 leverages a large language model (LLM) that exploits the data from the knowledge graph 50 into downstream tasks such as generating a summary of information from the knowledge graph 50 that was requested by a natural language query 402. Here, the natural language query 402 may be provided as a prompt to the LLM 400, whereby the LLM 400 is conditioned on the knowledge graph 50 to generate the response 404 that conveys the information requested by the prompt query 402. The user may provide follow-up natural language queries 402 as follow-up prompts to the LLM 400 to further refine previous responses 404 output by the LLM 400 to provide a conversational interface. As such, the user interface and the inference model 400 may provide conversational assistant capabilities (e.g., chat bot) to allow the user to interact with the knowledge graph 50 using natural dialog.

Additionally or alternatively, the inference model 400 may include a neural network model that is trained to make predictions by traversing the knowledge graph 50. Here, the user 102 may provide the query 402 “Is it safe for an individual to take Drug X while taking Drug Y?” and the inference model 400 may convert the natural language query into a graph query to traverse the knowledge graph 50 to identify adverse event nodes having edges/links connected to drug nodes for Drug X and Drug Y. The training data used to train the neural network model 400 may include example training queries each paired with the knowledge graph 50 and ground-truth adverse event nodes (or other types of nodes of interest in the knowledge graph 50) to teach the neural network model 400 to learn how to convert the training query into a graph query to traverse the knowledge graph 50 and identify the corresponding ground-truth adverse event nodes paired with each training query. Other example natural language queries 402 may include “Can drug X cause adverse effect E for a patient B who is on drug Y”, “What is the risk for patient B to take drug X while on Drug Y”, or “What is the risk of patient B to take drug X while having co-morbidities C”. The inference model 400 may run inferences from the data associated with the identified nodes to make predictions regarding the safety of taking Drug X while taking Drug Y. As the nodes in the knowledge graph 50 may include embeddings in an embedding space, the inference model 400 may make predictions based on relationships between the nodes represented in the embedding space. These inferences may consider how many cases involve an individual taking both Drug X and Drug Y and a seriousness of any adverse events. The inferences may also consider adverse events related to other drugs that have similar characteristics to Drug X and Drug Y, such as drugs targeting similar biological pathways as Drugs X and Y, when running inferences to predict the safety of taking Drug X while taking Drug Y. These inferences may also identify unique characteristics in responses 404 output from the inference model 400 such as the knowledge graph 50 revealing that patients under 18 years old treated with both Drug X and Drug Y are very likely to experience a particular adverse event while patients over 60 years of age have not experienced any serious adverse events. Based on the predictions, the inference model 400 may generate one or more candidate responses to the query 402, and may optionally score the candidate responses based on the knowledge graph 50. The inference model 400 may present the best scoring candidate response to the user via the UI 500 or may present all or just a few of the top scoring candidate responses to the user 10 via the UI 500.

Referring to FIGS. 4 and 5A, in some configurations, the inference model 400 receives pre-configured queries 402 from the user device 110 in response to user input indications indicating selection of menu items, graphical features, and/or filter options presented in the UI 500. The UI 500, 500a of FIG. 5A may correspond to a dashboard or reporting tool for accessing and viewing information associated with the knowledge graph 50. Additionally, the UI 500 may allow the user 110 to input natural language queries 402 into a text field 502 presented in the UI 500. As such, the user 102 may issue a natural language query 402 and then further refine a search for what information the user 102 wants to retrieve, or have the inference model 400 infer, from the knowledge graph 50 by issuing pre-configured queries 402 through selection of graphical elements 504 such as, without limitation, menu items, dropdowns, and/or filtering options presented in the UI 500.

FIG. 5A shows the UI 500, 500a permits the user 102 to interact with the knowledge graph 50 by allowing the user 102 to issue one or more queries 402 specifying information associated with the knowledge graph 50 and then present the information associated with the knowledge graph 50 that was specified by the queries 402. For instance, the UI 500a may present the information retrieved from the knowledge graph 50 in a form easy for the user 102 to view by populating a table 520 with the information retrieved from the knowledge graph 50. In the example shown, the table 520 includes a number of rows each associated with a respective case of a patient/participant prescribed a particular drug (e.g., C5013) and columns including values obtained from the knowledge graph 50 for various attributes such as demographics (e.g., gender/age) of each patient/participant, any con-meds the patients/participants are taking, patient/participant risk/factors, adverse events, drug labels, and case narratives. The values populated into each column of the table 520 may include information ascertained from the nodes of the knowledge graph 50. Moreover, some of the columns may be populated with hyperlinks to information sources that the user 102 may select to be directed to the information sources. For example, the user 102 may view a case narrative for a respective one of the patients/participants by selecting the “View” hyperlink presented in the “Narratives” column. In this example, the UI 500 may display a webpage that includes the case narrative. The UI 500 may be configured to present the webpage as a pop-up viewer overtop the table 520 so that the user 102 can scan through the case narrative without being directed away from the table 520.

Referring to FIG. 5B, in some implementations, the UI 500, 500b presents an interactive knowledge graph 50. In the example shown, the knowledge graph 50 includes a disease node (e.g., cancer or a particular type of cancer) as a root node and treatment nodes 1-3 branching off of the disease node that each correspond to a different type of treatment for treating the disease associated with the disease node. Here, a first treatment node (Treatment 1) may include a first type of treatment such as immunotherapy, a second treatment node (Treatment 2) may correspond to a second type of treatment such as hormone therapy, and a third treatment node (Treatment 3) may correspond to a third type of treatment such as chemotherapy. While not shown, any one of the treatment nodes may also be connected to one or more other disease nodes indicating that the corresponding type of treatment may be used to treat more than one disease or multiple different types of a disease. For simplicity, the knowledge graph 50 only depicts the nodes branching from, and related to, the first treatment node (Treatment 1).

The knowledge graph 50 shows a number of drug nodes (Drug 1, Drug 2, . . . . Drug N) branching off from the first treatment node (Treatment 1) that each correspond to a different drug associated with the first type of treatment for treating the underlying disease. One or more of the drugs represented by the drug nodes may include investigational drugs that have been evaluated in clinical trials for treating the disease. Additionally or alternatively, one or more of the drugs represented by the drug node may include drugs that have been approved by a regulatory authority (e.g., FDA) as effective for treating the disease. For simplicity, the knowledge graph 50 only depicts child nodes branching from, and related to, the first drug (Drug 1).

Branching from the first drug node (Drug 1), the knowledge graph 50 includes a number of adverse event nodes (AE 1, AE 2, AE 3) that each correspond to a respective adverse event related to the first drug node (Drug 1) associated with the first type of treatment (Treatment 1) for treating the underlying disease. In some examples, the adverse events indicated by the AE nodes include preferred terms (PTs) as specified by the MedDRA directory. The interactive knowledge graph 500b may further present detailed information for a given adverse event node such as related terms, synonymous terms, and lexical variants for the PT responsive to receiving a user input indication indicating selection of the given adverse event node displayed in the interactive knowledge graph 500b. Optionally, the knowledge graph 50 may include a pathway node branching from the first drug nodes that indicates the biological pathway related to the first drug node (Drug 1). While not shown in the example, additional edges may connect the same pathway node to other drug nodes associated with the same or different treatment nodes of the interactive knowledge graph 50. In some examples, the user 102 provides a refinement query 402 (i.e., a natural language query or pre-configured query) that requests the interactive knowledge graph 50 to selectively present or remove a specific type of node such as pathway nodes. Similarly, the refinement query 402 can be more granular where the user 102 can instruct the interactive knowledge graph 50 to only depict a particular type of node branching from an identified source node (e.g., a query 402 to present only AE nodes branching from an identified drug node without presenting the AE nodes branching from the other drug nodes). In some examples, the user 102 interacts with the interactive knowledge graph 50 by providing a user input indication indicating selection of a particular node of the knowledge graph 50, thereby causing the interactive knowledge graph 50 to present child nodes that branch from the particular node selected by the user 102. The interactive knowledge graph 50 may receive a user input indication through the use of an input device such as, without limitation, touch input when the display 118 includes a touch screen, a mouse or stylist, image capture devices recognizing gestures and/or gaze direction, or a speech interface.

Branching from the first AE node (AE 1), the knowledge graph 50 includes a first group of one or more patient nodes (Patients A) that each indicate a respective patient/participant that experienced the first adverse event during or after treatment of the drug represented by the first drug node. Branching from the second AE node (AE 2), the knowledge graph 50 includes a second group of one or more patient nodes (Patients B) that each indicate a respective patient/participant that experienced the second adverse event during or after treatment of the drug represented by the first drug node. The first group of one or more patient nodes (Patients A) also branch from the second AE node (AE 2) indicating each respective patient/participant experienced both the second adverse event and the first adverse event during or after treatment of the drug represented by the first drug node. In the example shown, the first group of patient nodes (Patients A) may form a first cluster (e.g., in the embedding space) based on the respective patients/participants sharing a first trait/characteristic and the second group of patient nodes (Patients B) may form a second cluster (e.g., in the embedding space) based on the respective patients/participants sharing a second trait/characteristic that is different than the first train/characteristic. To illustrate by way of example, the first AE node (AE 1) may indicate the adverse event of hair loss, the second AE node (AE 2) may indicate the adverse event of hypotension, each respective patient/participant represented by the first group of patient nodes (Patients A) is a female (e.g., first characteristic/trait), and each respective patient/participant represented by the second group of patient nodes (Patients B) is a male (e.g., second characteristic/trait). Here, the interactive knowledge graph 50 may reveal to the user 102 that females taking the first drug (Drug 1) will experience hair loss as an adverse event while males who take the first drug (Drug 1) will not experience hair loss. Yet, both the female patients/participants represented by the first group of patient nodes (Patients A) and the male patients/participants represented by the second group of patients nodes (Patients B) who take the first drug (Drug 1) will experience hypertension independent.

As described above in the preceding paragraphs, the knowledge graph builder 200 may determine an embedding value for each of the nodes and construct the knowledge graph 50 by presenting the nodes in the embedding space such that nodes closer to one another within the embedding space are more related than nodes that are farther from one another in the embedding space. Accordingly, the length (and optionally the direction) of an edge connecting two nodes may indicate how related the two nodes are to one another. In a non-limiting example, if the training healthcare data 300T indicates that substantially every patient/participant who took a particular drug experienced a particular adverse event, then a length of an edge connecting the drug node and the adverse event node would be shorter than if only a small portion of those patients/participants experienced the particular adverse event. By way of example, the knowledge graph 50 contains nodes representing input taxicogenomics data to understand diseases, targets, drugs, and adverse events. The knowledge graph 50 leverages machine learning to compute edges between the nodes to help predict potential adverse events.

With continued reference to FIG. 5B, the knowledge graph 50 additionally includes a third group two patient nodes (Patients C) branching from the third AE node (AE 3) that represent respective patients/participants that experienced the third adverse event during or after treatment of the drug represented by the first drug node. In this example, the third adverse event may be a fatal adverse event such as circulatory collapse that resulted in death of both of the patents/participants represented by the third group of two patient nodes (Patients C). Based on the long length of the edge connecting the first drug node (Drug 1) to the third adverse event node (AE 3) and the fact that only two patients/participants suffered the adverse event, the interactive knowledge graph 50 presented by the UI 500b of FIG. 5B may deem the third adverse event (e.g., circulatory collapse) as a rare event that may occur in patients/participants who take the first drug represented by the first drug node (Drug 1). Yet, the UI 500b of FIG. 5B may allow the user 102 to run inferences on the knowledge graph 50 to ascertain a possible cause of the third adverse event. Here, the user 102 may issue a query 402 that requests the inference model 400 to identify any common characteristics shared by the two patients/participants represented by the third group of two patient nodes in the interactive knowledge graph 50 but not shared by a majority of the patients/participants represented by the first and second groups of patient nodes in the interactive knowledge graph 50. The inference model 400 may traverse the nodes of the interactive knowledge graph 50 and determine that both of the patients represented by the third group of two patient nodes (Patients C) also took a concomitant medication with the first drug that none of the other participants/patients represented by the other groups of patient nodes (Patients A and B) took. The inference model 400, via the UI 500b, could present a summary of this finding and provide a link to the drug label for the concomitant medication. Upon review of the drug label, the user 102 may learn that circulatory collapse is a known adverse event of the concomitant medication. As described in the remarks above, the user may issue natural language queries 402 to the inference model 400 via the UI 500b and the inference model 400 may leverage a LLM 400 to return a response 404 that summarizes information contained in the interactive knowledge graph 50 responsive to a query 402. The response 404 may annotate the summarized information with appropriate links that the user may select to ascertain additional information.

The interactive knowledge graph 50 may present detailed information related to a node when the interactive knowledge graph 50 receives a user input indication indicating selection of the node. For instance, the user 102 may select one of the patient nodes to cause the interactive knowledge graph 50 to present detailed information for the patient represented by the selected patient node. The interactive knowledge graph 50 may display a pop-up window that conveys the detailed information. The detailed information may include the patient's demographic information, details of a clinical trial the patient participated in, con-meds the patient took while taking the first drug, all adverse events experienced by the patient, and any other type of information available to the knowledge graph 50 that may be of interest. The interactive knowledge graph 50 may further annotate some of the detailed information such as by providing hyperlinks to sources of the detailed information. For instance, the interactive knowledge graph 50 may provide at least one of a hyperlink to the clinical trial the patient participated in, a hyperlink to lab results or an electronic medical record (EMR) for the patient, or a hyperlink to drug labels for the first drug and any con-meds the patient took while taking the first drug.

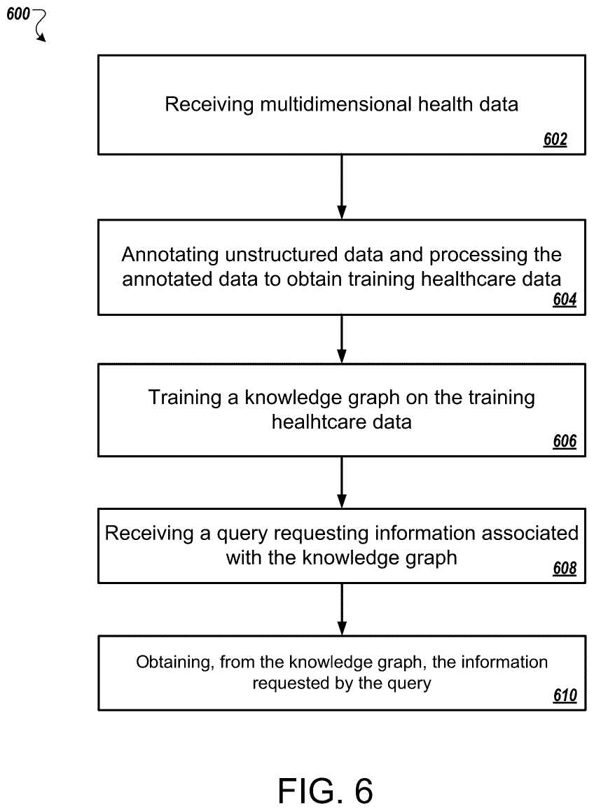

FIG. 6 is a flowchart of an example arrangement of operations for a method 600 of creating a knowledge graph 50 from multidimensional health data 300 and running an inference on the knowledge graph 50. The data processing hardware 142 of FIG. 1 may execute instructions stored on the memory hardware 144 of FIG. 1 that causes the data processing hardware 142 to perform the operations for the method 600. At operation 602, the method 600 includes receiving the multidimensional health data 300 from at least one data source 202. Here, the multidimensional health data includes unstructured data 300u. The multidimensional health data 300 may also include structured data 300b. At operation 604, the method 300 includes annotating the unstructured data 300u to generate annotated data 300a and processing the annotated data 300a to obtain training healthcare data 300T.

At operation 606, the method 600 includes training a knowledge graph 50 on the training healthcare data 300T. At operation 608, the method 600 includes receiving a query 402 requesting information associated with the knowledge graph 50. At operation 610, the method 600 includes obtaining, from the knowledge graph 50, the information requested by the query 402. The query 402 may be received from a user device 110 associated with a user 102 and the method 600 may transmit/provide a response 404 to the user device 110 that conveys the information obtained from the knowledge graph 50.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

The non-transitory memory may be physical devices used to store programs (e.g., sequences of instructions) or data (e.g., program state information) on a temporary or permanent basis for use by a computing device. The non-transitory memory may be volatile and/or non-volatile addressable semiconductor memory. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

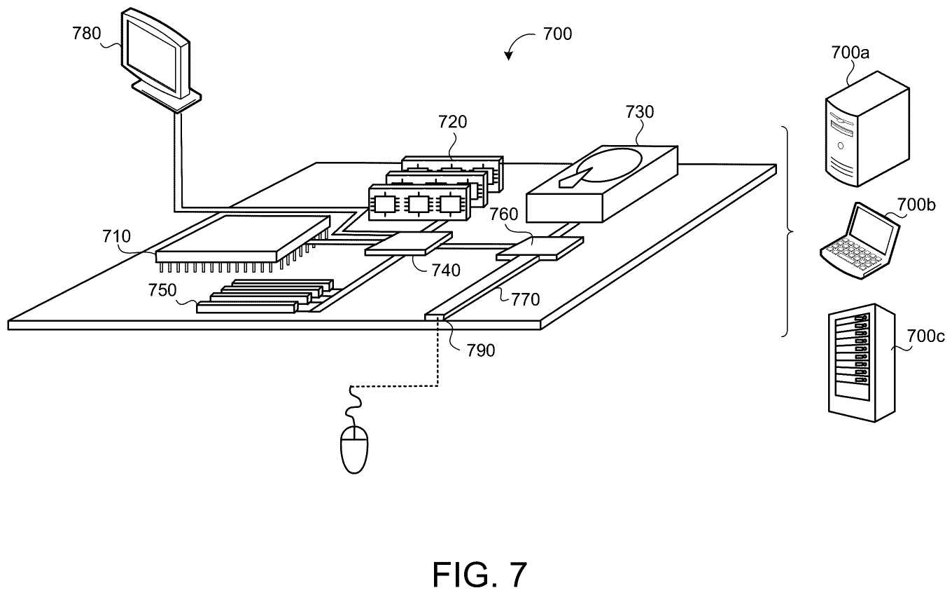

FIG. 7 is schematic view of an example computing device 700 that may be used to implement the systems and methods described in this document. The computing device 700 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 700 includes a processor 710, memory 720, a storage device 730, a high-speed interface/controller 740 connecting to the memory 720 and high-speed expansion ports 750, and a low speed interface/controller 760 connecting to a low speed bus 770 and a storage device 730. Each of the components 710, 720, 730, 740, 750, and 760, are interconnected using various busses, and may be mounted on a common motherboard or in other manners as appropriate. The processor 710 can process instructions for execution within the computing device 700, including instructions stored in the memory 720 or on the storage device 730 to display graphical information for a graphical user interface (GUI) on an external input/output device, such as display 780 coupled to high speed interface 740. In other implementations, multiple processors and/or multiple buses may be used, as appropriate, along with multiple memories and types of memory. Also, multiple computing devices 700 may be connected, with each device providing portions of the necessary operations (e.g., as a server bank, a group of blade servers, or a multi-processor system).

The memory 720 stores information non-transitorily within the computing device 700. The memory 720 may be a computer-readable medium, a volatile memory unit(s), or non-volatile memory unit(s). The non-transitory memory 720 may be physical devices used to store programs (e.g., sequences of instructions) or data (e.g., program state information) on a temporary or permanent basis for use by the computing device 700. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 730 is capable of providing mass storage for the computing device 700. In some implementations, the storage device 730 is a computer-readable medium. In various different implementations, the storage device 730 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 720, the storage device 730, or memory on processor 710.

The high speed controller 740 manages bandwidth-intensive operations for the computing device 700, while the low speed controller 760 manages lower bandwidth-intensive operations. Such allocation of duties is exemplary only. In some implementations, the high-speed controller 740 is coupled to the memory 720, the display 780 (e.g., through a graphics processor or accelerator), and to the high-speed expansion ports 750, which may accept various expansion cards (not shown). In some implementations, the low-speed controller 760 is coupled to the storage device 730 and a low-speed expansion port 790. The low-speed expansion port 790, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 700 may be implemented in a number of different forms, as shown in the figure. For example, it may be implemented as a standard server 700a or multiple times in a group of such servers 700a, as a laptop computer 700b, or as part of a rack server system 700c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. A computer-implemented method executed on data processing hardware that causes the data processing hardware to perform operations comprising:
receiving multidimensional health data from at least one data source, the multidimensional health data comprising unstructured data;
annotating the unstructured data to generate annotated data;
processing the annotated data to obtain training healthcare data;
training a knowledge graph on the training healthcare data;
receiving a query requesting information associated with the knowledge graph; and
obtaining, from the knowledge graph, the information requested by the query.

2. The computer-implemented method of claim 1, wherein:
the query comprises a natural language query; and
obtaining the information requested by the query comprises:
processing, using an inference model, the natural language query by performing query interpretation on the natural language query to determine a type of the information requested by the natural language query; and
based on the type of the information requested by the natural language query, retrieving the information from the knowledge graph.

3. The computer-implemented method of claim 2, wherein the operations further comprise:
generating, using the inference model, a natural language summary of the information retrieved from the knowledge graph; and
providing the natural language summary of the information for output from a user device.

4. The computer-implemented method of claim 3, wherein the inference model leverages a large language model to generate the natural language summary of the information.

5. The computer-implemented method of claim 2, wherein the inference model comprises a neural network model.

6. The computer-implemented method of claim 1, wherein the operations further comprise:
receiving canonical reference data,
wherein annotating the unstructured data comprises annotating the unstructured data based on the canonical reference data.

7. The computer-implemented method of claim 1, wherein the operations further comprise:
receiving concepts that define an ontology for semantically linking the training healthcare data,
wherein training the knowledge graph on the training healthcare data comprises using the concepts to train the knowledge graph on the training healthcare data.

8. The computer-implemented method of claim 1, wherein the operations further comprise executing a knowledge controller, the knowledge controller configured to display, on a screen of a user device, a user interface for viewing the information obtained from the knowledge graph.

9. The computer-implemented method of claim 8, wherein receiving the query comprises receiving the query from the user device, the query input by the user through the user interface.

10. The computer-implemented method of claim 1, wherein the operations further comprise:
executing a knowledge controller, the knowledge controller configured to display, on a screen of a user device, a user interface; and
displaying, in the user interface, the knowledge graph as an interactive knowledge graph.

11. The computer-implemented method of claim 1, wherein the information requested by the query comprises information regarding a safety of a specific drug for treating a disease.

12. A system comprising:
data processing hardware; and
memory hardware in communication with the data processing hardware and storing instructions that when executed by the data processing hardware causes the data processing hardware to perform operations comprising:
receiving multidimensional health data from at least one data source, the multidimensional health data comprising unstructured data;
annotating the unstructured data to generate annotated data;
processing the annotated data to obtain training healthcare data;
training a knowledge graph on the training healthcare data;
receiving a query requesting information associated with the knowledge graph; and
obtaining, from the knowledge graph, the information requested by the query.

13. The system of claim 12, wherein:
the query comprises a natural language query; and
obtaining the information requested by the query comprises:
processing, using an inference model, the natural language query by performing query interpretation on the natural language query to determine a type of the information requested by the natural language query; and
based on the type of the information requested by the natural language query, retrieving the information from the knowledge graph.

14. The system of claim 13, wherein the operations further comprise:
generating, using the inference model, a natural language summary of the information retrieved from the knowledge graph; and
providing the natural language summary of the information for output from a user device.

15. The system of claim 14, wherein the inference model leverages a large language model to generate the natural language summary of the information.

16. The system of claim 13, wherein the inference model comprises a neural network model.

17. The system of claim 12, wherein the operations further comprise:
receiving canonical reference data,
wherein annotating the unstructured data comprises annotating the unstructured data based on the canonical reference data.

18. The system of claim 12, wherein the operations further comprise:
receiving concepts that define an ontology for semantically linking the training healthcare data,
wherein training the knowledge graph on the training healthcare data comprises using the concepts to train the knowledge graph on the training healthcare data.

19. The system of claim 12, wherein the operations further comprise executing a knowledge controller, the knowledge controller configured to display, on a screen of a user device, a user interface for viewing the information obtained from the knowledge graph.

20. The system of claim 19, wherein receiving the query comprises receiving the query from the user device, the query input by the user through the user interface.

21. The system of claim 12, wherein the operations further comprise:
executing a knowledge controller, the knowledge controller configured to display, on a screen of a user device, a user interface; and
displaying, in the user interface, the knowledge graph as an interactive knowledge graph.

22. The system of claim 12, wherein the information requested by the query comprises information regarding a safety of a specific drug for treating a disease.
